(19) United States
(12) Patent Application Publication
(10) Pub. No.: US 2024/0306950 A1
(43) Pub. Date: Sep. 19, 2024

(54) SYSTEM AND METHOD FOR MONITORING HEALTH PARAMETERS

(71) Applicant: KNOW LABS, INC., Seattle, WA (US)

(72) Inventor: JOHN CRONIN, Seattle, WA (US)

(21) Appl. No.: 18/601,451

(22) Filed: Mar. 11, 2024

Related U.S. Application Data
(60) Provisional application No. 63/490,643, filed on Mar. 16, 2023.

(51) Int. Cl. A61B 5/145; A61B 5/00; A61B 5/01; A61B 5/05; A61B 5/11; G01K 13/20

(52) U.S. Cl. CPC A61B 5/14532; A61B 5/0022; A61B 5/01; A61B 5/05; A61B 5/11; A61B 5/14546; G01K 13/20; A61B 2560/0252

(57) ABSTRACT

A method for monitoring a signal that corresponds to blood glucose level. The method includes transmitting by one or more transmitting (TX) antennas, active RF radio waves below the skin surface of a user, and receiving radio waves on one or more receiving (RX) antennas. The received radio waves include a response portion of the transmitted radio waves. Further, the method includes collecting signals in response to receiving the radio waves on one or more RX antennas; modifying the signals to filter out certain frequencies bands not associated with glucose waveforms; sending the signals to a network processing module; and receiving a blood glucose level from the network processing module in response to the signals.

### FIELD

The present disclosure is generally related to systems and methods of monitoring health parameters and, more particularly, relates to a system and a method of monitoring a signal that corresponds to the blood glucose level in a user.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Diabetes is a medical condition in which a person's blood glucose level, also known as blood sugar level, is persistently elevated. Diabetes can result in severe medical complications, including cardiovascular disease, kidney disease, stroke, foot ulcers, and eye damage if left untreated. Typically, diabetes is caused by either insufficient insulin production by the pancreas, referred to as “Type 1 diabetes,” or improper insulin response by the body's cells, referred to as “Type 2 diabetes.” Further, monitoring a person's blood glucose level and administering insulin when a person's blood glucose level is too high to reach a desired level may be a part of managing diabetes. Depending on many factors, such as the severity of diabetes and the individual's medical history, a person may need to measure their blood glucose level up to ten times per day. Each year, billions of dollars are spent on equipment and supplies for monitoring blood glucose levels.

Moreover, regular glucose monitoring is a crucial component of diabetes care. Further, measuring blood glucose is generally an invasive procedure by giving a blood sample at a clinic or hospital. Home glucose monitoring is also possible using a variety of devices. The blood sample is obtained by pricking the skin using a tiny instrument. A glucose meter or glucometer is a tiny instrument that measures the sugar in the blood sample. The majority of glucose monitoring methods and devices require a blood sample.

Currently, available glucose monitoring devices also require a blood sample, usually by pricking the skin and then using a polling technique to determine the glucose level of a patient. These monitoring devices are almost 95 percent accurate and are also preferable. However, such monitoring devices are often prone to contamination as the patient may not be in standard conditions to give the blood sample.

### SUMMARY

A system and method to monitor glucose levels with enhanced accuracy and without requiring a blood sample from the patient. A monitored signal corresponds to blood glucose level. The method includes transmitting by one or more transmitting (TX) antennas, active RF radio waves below the skin surface of a user, and receiving radio waves on one or more receiving (RX) antennas. The received radio waves include a response portion of the transmitted radio waves. Further, the method includes collecting signals in response to receiving the radio waves on one or more RX antennas; modifying the signals to filter out certain frequencies bands not associated with glucose waveforms; sending the signals to a network processing module; and receiving a blood glucose level from the network processing module in response to the signals.

One example of a health monitoring system can include a monitoring device that includes one or more transmit antennas configured to transmit radio-frequency (RF) analyte detection signals into a user and one or more receive antennas configured to detect RF analyte signals that result from the RF analyte detection signals transmitted into the user. An analog-to-digital converter is connected to the one or more receive antennas and receives the RF analyte signals detected by the one or more receive antennas. In addition, the system includes a motion sensor that detects motion of the user during transmission of the RF analyte detection signals by the one or more transmit antennas and during detection of the RF analyte signals by the one or more receive antennas, and/or a body temperature sensor that detects the temperature of the user (and/or environmental temperature) during transmission of the RF analyte detection signals by the one or more transmit antennas and during detection of the RF analyte signals by the one or more receive antennas.

Another example of a health monitoring system can include a monitoring device that includes one or more transmit antennas configured to transmit radio-frequency (RF) analyte detection signals into a user and one or more receive antennas configured to detect RF analyte signals that result from the RF analyte detection signals transmitted into the user. An analog-to-digital converter is connected to the one or more receive antennas and receives the RF analyte signals detected by the one or more receive antennas. The monitoring device can also include a motion sensor that detects motion of the user during transmission of the RF analyte detection signals by the one or more transmit antennas and during detection of the RF analyte signals by the one or more receive antennas.

Another example of a health monitoring system can include a monitoring device that includes one or more transmit antennas configured to transmit radio-frequency (RF) analyte detection signals into a user and one or more receive antennas configured to detect RF analyte signals that result from the RF analyte detection signals transmitted into the user. An analog-to-digital converter is connected to the one or more receive antennas and receives the RF analyte signals detected by the one or more receive antennas. In addition, a body temperature sensor detects the temperature of the user during transmission of the RF analyte detection signals by the one or more transmit antennas and during detection of the RF analyte signals by the one or more receive antennas.

One example of a health monitoring method can include detecting an analyte in a user by transmitting radio-frequency (RF) analyte detection signals into the user from one or more transmit antennas and detecting, using one or more receive antennas, RF analyte signals that result from the RF analyte detection signals transmitted into the user. The method also includes converting the detected RF analyte signals from analog signals to digital signals using an analog-to-digital converter connected to the one or more receive antennas. In addition, the method includes one or more of: a) during transmission of the RF analyte detection signals by the one or more transmit antennas and during detection of the RF analyte signals by the one or more receive antennas, sensing motion of the user using a motion sensor; b) during transmission of the RF analyte detection signals by the one or more transmit antennas and during detection of the RF analyte signals by the one or more receive antennas, sensing a body temperature (and/or environmental temperature) of the user using a body temperature sensor (and/or an environmental temperature detector).

### DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a block diagram of a system for monitoring the health parameters of a user, according to an embodiment;

FIG. 2 illustrates a posterior view of a hand of the user with an approximate location of a cephalic vein and a basilic vein overlaid/superimposed, according to an embodiment;

FIGS. 3A-3B illustrate cross-sectional views of a wrist showing the ulna bone and the basilic vein with FIG. 3B illustrating a monitoring device described herein attached to the wrist, according to an embodiment;

FIG. 4 illustrates an example of a prior art functional block diagram of the system.

FIG. 5 illustrates a circuitry layout of the system on a substrate, according to an embodiment;

FIG. 6 illustrates the circuitry layout of the system overlaid on the wrist of the user, according to an embodiment;

FIGS. 7A-7B illustrate a flowchart of a method executed on a device base module, according to an embodiment;

FIG. 8 illustrates a flowchart of a method performed by a network base module, according to an embodiment; and

FIG. 9 illustrates a flowchart of a method performed by a network processing module, according to an embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

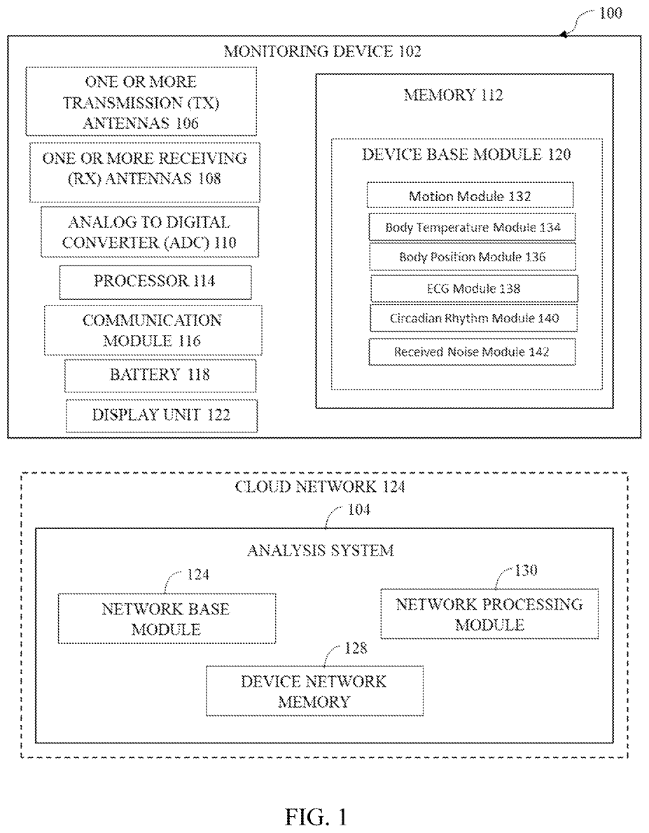

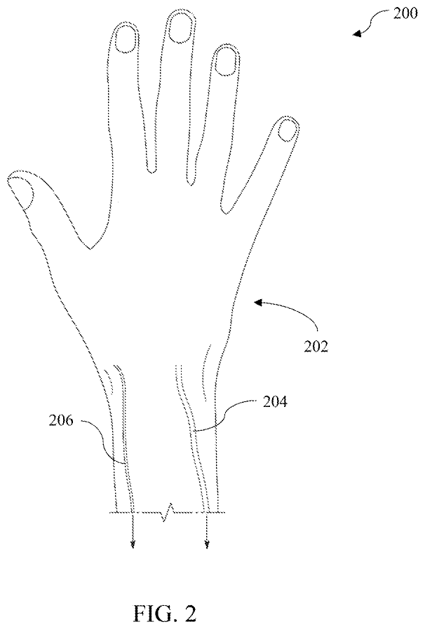

FIG. 1 illustrates a block diagram of a system 100 for monitoring the health parameters of a user, according to an embodiment. FIG. 1 is described in conjunction with FIGS. 2-9.

The system 100 may comprise a monitoring device 102 communicatively coupled to a device network 104. In one embodiment, the device network 104 may be a wireless and/or wired communication channel. Device network 104 is contained in one or more servers integrated into the internet in one embodiment. The monitoring device 102 may be worn by the user. The monitoring device 102 may be configured to collect signals in response to receiving radio frequency signals of RF range. The monitoring device 102 may target the active RF radio frequency signals to blood in blood vessels, and response signals may correspond to the blood glucose level in the user. It should be noted that the response signals are signals received by the RX antenna with a time window of a transmitted TX RF signal. US 2020/0187836 is incorporated herein by reference in its entirety.

In one embodiment, the system 100 may include integrated circuit (IC) devices (not shown) with transmit and/or receive antennas integrated therewith. Collecting signals in response to receiving radio frequency signals from the specific blood vessels of the user involves the transmission of suitable active RF radio frequency signals below the user's skin surface. Corresponding to the transmission, a response portion of the active RF radio frequency signals is received on multiple receive antennas. Further, the monitoring device 102 may collect the signals in response to receiving the radio waves on the multiple receive antennas. The monitoring device 102 may further output a signal from the received active RF radio frequency signals that correspond to the blood glucose level in the user. It can be noted that the monitoring device 102 may be worn by the user at various locations such as wrist, arm, leg, etc. Further, the system 100 may process the collected signals to determine the user's health parameters. The health parameters of the user may include blood pressure (mmHg), glucose level (mg/dL), oxygen level (%), etc.

In one embodiment, the system 100 for monitoring the blood glucose level of the user using the active RF radio frequency signals involves transmitting active RF radio frequency signals below the skin surface, receiving a response portion of the active RF radio frequency signals on multiple receive antennas, collecting a signal in response to receiving the radio waves on the multiple receive antennas, outputting a signal with filtered frequency waveform that corresponds to the blood glucose waveform and processing the filtered collected signal to determine blood glucose level of the user. In one embodiment, beamforming is used for modifying the radio frequency signal received from the receiving antenna. In the beamforming process, the certain frequency band not associated with glucose waveforms is eliminated. In another embodiment, Doppler effect processing may be used to modify the radio frequency signal received from the receiving antenna to determine the signal with filtered frequency waveform. It can be noted that analog and/or digital signal processing techniques may be used to implement beamforming and/or Doppler effect processing and digital signal processing of the received signals to dynamically adjust a received beam onto the desired location. In another embodiment, the beamforming and the Doppler effect processing may be used together to modify the radio frequency signal received from the receiving antenna.

In one exemplary embodiment, active RF radio frequency signals of a higher frequency range of 122-126 gigahertz (GHz) with a shallower penetration depth are used to monitor blood glucose levels. It can be noted that the shallower penetration depth reduces undesirable reflections, such as reflections from bone, and dense tissue, such as tendons, ligaments, and muscle, which may reduce the signal processing burden and improve the quality of the desired signal generated from the location of the blood vessel. It can also be noted that bones are dielectric and semi-conductive. In addition, bones are anisotropic, so not only are bones conductive, and they conduct differently depending on the direction of the flow of current through the bone. Alternatively, the bones are also piezoelectric materials. Therefore, an example radio frequency signals of a higher frequency range of 122-126 GHz with a shallower penetration depth may be required to monitor the blood glucose levels. There are many embodiments of signals in the RF range from 500 MHZ to 300 GHZ, either as a single frequency, or a range of frequencies, or even combinations of frequencies can be used to monitor the blood in the blood vessels.

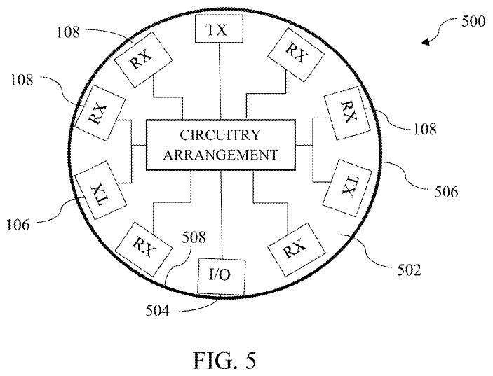

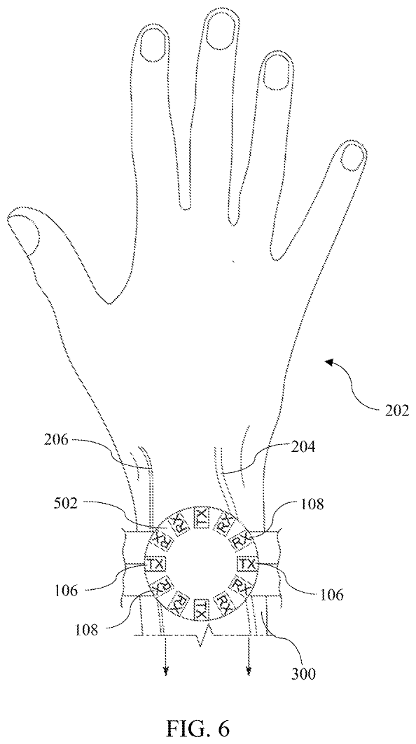

Further, the monitoring device 102 may comprise one or more transmission (TX) antennas 106, one or more receiving (RX) antennas 108, an analog to digital converter (ADC) 110, a memory 112, a processor 114, a communication module 116, a battery 118 and a device base module 120. In one embodiment, the monitoring device 102 may be a wearable and portable device such as, but not limited to, a cell phone, a smartwatch, a tracker, a wearable monitor, a wristband, and a personal blood monitoring device. The one or more TX antennas 106 and the one or more RX antennas 108 may be fabricated over a substrate (not shown) within the monitoring device 102 in a suitable configuration. In one exemplary embodiment, at least two TX antennas and at least four RX antennas are fabricated over the substrate. The one or more TX antennas 106 and the one or more RX antennas 108 may correspond to a circuitry arrangement (not shown) over the substrate. The circuitry arrangement on the substrate is described in the later part of the detailed description in conjunction with FIGS. 5-6. Further, the ADC 110, the memory 112, the processor 114, the communication module 116, the battery 118, the device base module 120, and a display unit 122 may be fabricated over the substrate. Further, the communication module 116 may be configured to facilitate communication between the monitoring device 102 and the device network 104.

Further, the one or more TX antennas 106 and the one or more RX antennas 108 may be integrated into the circuitry arrangement. The one or more TX antennas 106 may be configured to transmit the active RF radio frequency signals at a pre-defined frequency. In one embodiment, the pre-defined frequency may correspond to a range suitable for the human body. Successively, the one or more RX antennas 108 may be configured to receive the response portion of the active RF radio frequency signals.

In one embodiment, the active RF radio frequency signals may be transmitted under the user's skin, and electromagnetic energy may be a response from many body parts such as fibrous tissue, muscle, tendons, bones, and the skin. It can be noted that effective monitoring of the blood glucose level is facilitated by an electrical response of blood molecules, such as pancreatic endocrine hormones, against the transmitted active RF radio frequency signals. It will be apparent to a skilled person that the pancreatic endocrine hormones such as insulin and glucagon are responsible for maintaining sugar or glucose level. Further, the electromagnetic energy response from the blood molecules may be received by the one or more RX antennas 108. Further, the ADC 110 may be coupled to the one or more RX antennas 108. The one or more RX antennas 108 may be configured to receive the response active RF radio frequency signals. The ADC 110 may be configured to convert the active RF radio frequency signals from an analog signal into a digital processor readable format. It should be noted that TX and RX antennas 106 and 108 may be on a separate substrate and not within monitoring device 106 in one embodiment.

Further, the memory 112 may be configured to store the transmitted active RF radio frequency signals by the one or more TX antennas 106 and receive a response portion of the transmitted active RF radio frequency signals from the one or more RX antennas 108. Further, the memory 112 may also store the converted digital processor readable format by the ADC 110. In one embodiment, the memory 112 may include suitable logic, circuitry, and/or interfaces that may be configured to store a machine code and/or a computer program with at least one code section executable by the processor 114. Examples of implementation of the memory 112 may include, but are not limited to, Random Access Memory (RAM), Read Only Memory (ROM), Hard Disk Drive (HDD), and/or a Secure Digital (SD) card.

Further, the system 100 may comprise the device base module 120 fabricated within the memory 112. The device base module 120 may be configured to store instructions in memory 112 for executing the computer program from the converted digital processor readable format of the ADC 110. The device base module 120 is configured to facilitate the operation of the processor 114, the memory 112, the one or more TX antennas 106, the one or more RX antennas 108, and the communication module 116. Further, the device base module 120 may be configured to create polling of the active RF radio frequency signals.

Further, the processor 114 may facilitate the operation of the monitoring device 102 with the device network 104 to perform functions according to the instructions stored in the memory 112. In one embodiment, the processor 114 may include suitable logic, circuitry, interfaces, and/or code that may be configured to execute a set of instructions stored in the memory 112. The processor 114 may be configured to run the instructions obtained by the device base module 120 to perform polling. The processor 114 may be further configured to collect real-time signals to be transmitted by the one or more TX antennas 106 and received by the one or more RX antennas 108 and may store the real-time signals in the memory 112. In one embodiment, the processor is driven by code (not shown) in memory 112 to input the signals from the ADC on a data bus (not shown) and store these signals in memory 112. In one embodiment, the real-time signals may be assigned as initial and updated radio frequency (RF) signals.

In one embodiment, the processor 114 may be configured to modify the real-time signals received from the RX antennas 108. The processor 114 may be configured to filter out certain frequency bands which do not correspond to glucose waveforms. The processor 114 may compare the radio signals of all frequencies with a threshold waveform. The threshold waveform may be the frequencies that correspond to the blood glucose level. Further, the memory 112 may store the modified signals having frequency bands associated with the glucose waveforms.

Examples of the processor 114 may be an X86-based processor, a Reduced Instruction Set Computing (RISC) processor, an Application-Specific Integrated Circuit (ASIC) processor, a Complex Instruction Set Computing (CISC) processor, and/or other processors. The processor 114 may be a multicore microcontroller specifically designed to carry multiple operations based upon pre-defined algorithm patterns to achieve a desired result.

Further, the processor 114 may take inputs from the monitoring device 102 and retain control by sending signals to different parts of the monitoring device 102. The processor 114 may access a Random Access Memory (RAM) that is used to store data and other results created when the processor 114 is at work. It can be noted that the data is stored temporarily for further processing, such as filtering, correlation, correction, and adjustment. Moreover, the processor 114 carries out special tasks as programs that are pre-stored in the Read Only Memory (ROM). It can be noted that the special tasks carried out by the processor 114 indicate and apply certain actions which trigger specific responses.

Further, the communication module 116 of the monitoring device 102 may communicate with the device network 104 via a cloud network 124. Examples of the communication module 116 may include, but are not limited to, the internet, a cloud network, a Wireless Fidelity (Wi-Fi) network, a Wireless Local Area Network (WLAN), a Local Area Network (LAN). In one embodiment, various devices may be configured to have a communication module integrated over circuitry arrangement to connect with the device network 104 via various wired and wireless communication protocols, such as the cloud network 124. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols. In one embodiment, the battery 118 may be disposed over the substrate to power hardware modules of the monitoring device 102. The monitoring device 102 may be configured with a charging port (not shown) to recharge the battery 118. It can be noted that the charging of the battery 118 may be performed by wired or wireless means. In one embodiment, the battery 118 may include different models of lithium-ion batteries, such as CR1216, CR2016, CR2032, CR2025, CR2430, CR1220, CR1620, and CR1616.

The system 100 may include a motion module 132 that includes at least one sensor from the group of an accelerometer, a gyroscope, an inertial movement sensor, or other similar sensor. The motion module 132 may have its own processor or utilize the processor 114 to calculate the user's movement. Motion from the user will change the blood volume in a given portion of their body and the blood flow rate in their circulatory system. This may cause noise, artifacts, or other errors in the real-time signals received by the RX antennas 108. The motion module 132 may compare the calculated motion to a motion threshold stored in memory 112. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 132 may flag the RF signals collected at the time stamp corresponding to the motion as potentially inaccurate. In some embodiments, the motion module 132 may compare RF signal data to motion data over time to improve the accuracy of the motion threshold. The motion module 132 may alert the user, such as with an audible beep or warning or a text message or alert to a connected mobile device. The alert would signal the user that they are moving too much to get an accurate measurement. The motion module may utilize the communication module 116 to communicate with the device network 104 in order to inform the network processing module 130 of the calculated motion of the user that corresponds with the received RF signal data. In this manner, the motion module may be simplified to just collect motion data and allow the network processing module 130 to determine if the amount of motion calculated exceeds a threshold that would indicate the received RF signal data is too noisy to be relied upon for a blood glucose measurement.

The system 100 may include a body temperature module 134 that includes at least one sensor from the group of a thermometer, a platinum resistance thermometer (PRT), a thermistor, a thermocouple, or another temperature sensor. The body temperature module 134 may have its own processor or utilize the processor 114 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 108. The body temperature module 134 may compare the measured temperature to a threshold temperature stored in memory 112. For example, the environmental temperature threshold may be set at zero degrees Celsius because low temperatures can cause a temporary narrowing of blood vessels which may increase the user's blood pressure. When the measured temperature exceeds the threshold, the body temperature module 134 may flag the RF signals collected at the time stamp corresponding to the temperature as potentially being inaccurate. In some embodiments, the body temperature module 134 may compare RF signal data to temperature data over time to improve the accuracy of the temperature threshold. The body temperature module 134 may alert the user, such as with an audible beep or warning or a text message or alert to a connected mobile device. The alert would signal to the user that their body temperature or the environmental temperature is not conducive to getting an accurate measurement. The body temperature module 134 may utilize the communication module 116 to communicate with the device network 104 in order to inform the network processing module 130 of the measured user or environmental temperature that corresponds with the received RF signal data. In this manner, the body temperature module 134 may be simplified to just collect temperature data and allow the network processing module 130 to determine if the temperature measure exceeds a threshold that would indicate the received RF signal data is too noisy to be relied upon for a blood glucose measurement.

The system 100 may include a body position module 136 that includes at least one sensor from the group of an accelerometer, a gyroscope, an inertial movement sensor, or another similar sensor. The body position module 136 may have its own processor or utilize the processor 114 to estimate the user's position. The user's body position may change the blood volume in a given part of their body and the blood flow rate in their circulatory system. This may cause noise, artifacts, or other errors in the real-time signals received by the RX antennas 108. The body position module 136 may compare the estimated position to a body position threshold stored in memory 112. For example, the monitoring device 102 may be on the user's wrist, and the body position threshold may be based on the relative position of the user's hand to their heart. When a user's hand is lower than their heart, their blood pressure will increase, with this effect being more pronounced the longer the position is maintained. Conversely, the higher a user holds their arm above their heart, the lower the blood pressure in their hand. The body position threshold may include some minimum amount of time the estimated body position occurs. When the estimated position exceeds the threshold, the body position module 136 may flag the RF signals collected at the time stamp corresponding to the body position as potentially being inaccurate. In some embodiments, the body position module 136 may compare RF signal data to motion data over time to improve the accuracy of the body position threshold. The body position data may also be used to estimate variations in parameters such as blood pressure that corresponds to the body position data to improve the accuracy of the measurements taken when the user is in that position. The body position module 136 may alert the user, such as with an audible beep or warning or a text message or alert to a connected mobile device. The alert would signal to the user that their body position is not conducive to getting an accurate measurement. The body position module 136 may utilize the communication module 116 to communicate with the device network 104 in order to inform the network processing module 130 of the estimated body position data that corresponds with the received RF signal data. In this manner, the body temperature module 134 may be simplified to just collect temperature data and allow the network processing module 130 to determine if the body position exceeded a threshold that would indicate the received RF signal data is too noisy to be relied upon for a blood glucose measurement.

The system 100 may include an ECG module 138 that includes at least one electrocardiogram sensor. The ECG module 138 may have its own processor or utilize the processor 114 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 108. The ECG module 138 may compare the measured cardiac data to a threshold stored in memory 112. For example, the threshold may be a pulse above 160 bpm, as the increased blood flow volume may cause too much noise in the received RF signal data to accurately measure the blood glucose. When the ECG data exceeds the threshold, the ECG module 138 may flag the RF signals collected at the time stamp corresponding to the ECG data as potentially being inaccurate. In some embodiments, the ECG module 138 may compare RF signal data to ECG data over time to improve the accuracy of the ECG data threshold or to improve the measurement of glucose at a given point in the cycle between peak and minimum cardiac output. The ECG module 138 may alert the user, such as with an audible beep or warning or a text message or alert to a connected mobile device. The alert would signal to the user that their heart rate is not conducive to getting an accurate measurement or requires additional medical intervention. The ECG module 138 may utilize the communication module 116 to communicate with the device network 104 in order to inform the network processing module 130 of the measured ECG data that corresponds with the received RF signal data. In this manner, the ECG module 138 may be simplified to just collect ECG data and allow the network processing module 130 to determine if the ECG data exceeded a threshold that would indicate the received RF signal data is too noisy to be relied upon for a blood glucose measurement.

The system 100 may include a circadian rhythm module 140 that includes at least one sensor measuring actigraphy, wrist temperature, light exposure, and heart rate. The circadian rhythm module 140 may have its own processor or utilize the processor 114 to calculate the user's circadian health. Blood pressure follows a circadian rhythm in that it increases upon waking in the morning and decreases during sleeping at night. People with poor circadian health will often have higher blood pressure. These variations in blood pressure can cause noise, artifacts, or other errors or inaccuracies in the real-time signals received by the RX antennas 108. The circadian rhythm module 140 may compare the circadian data to a threshold stored in memory 112. For example, the threshold may be less than 6 hours of sleep in the last 24 hours. When the observed circadian health data exceeds the threshold, the circadian rhythm module 140 may flag the RF signals collected at the time stamp corresponding to circadian health as potentially being inaccurate or needing an adjustment to account for the expected increase in the user's blood pressure. In some embodiments, the circadian rhythm module 140 may compare RF signal data to sleep data over time to improve the accuracy of the circadian rhythm thresholds. The circadian rhythm module 140 may alert the user, such as with an audible beep or warning, or a text message or alert to a connected mobile device. The alert would signal to the user that their recent sleep patterns are not conducive to getting an accurate measurement. The circadian rhythm module 140 may utilize the communication module 116 to communicate with the device network 104 in order to inform the network processing module 130 of the measured circadian data that corresponds with the received RF signal data. In this manner, the circadian rhythm module 140 may be simplified to just collect circadian rhythm data and allow the network processing module 130 to determine if the measure exceeded a threshold that would indicate the received RF signal data is too noisy to be relied upon for a blood glucose measurement, or if an alternative transfer function should be used to compensate for the detected circadian health.

The system 100 may include a received noise module 142 that includes at least one sensor measuring background signals such as RF signals, Wi-Fi, and other electromagnetic signals that could interfere with the signals received by the RX antennas 108. The received noise module 142 may have its own processor or utilize the processor 114 to calculate the level of background noise being received. Background noise may interfere with or cause noise, artifacts, or other errors or inaccuracies in the real-time signals received by the RX antennas 108. The received noise module 142 may compare the level and type of background noise to a threshold stored in memory 112. The threshold may be in terms of field strength (volts per meter and ampere per meter) or power density (watts per square meter). For example, the threshold may be RF radiation greater than 300 μW/m2. When the background noise data exceeds the threshold, the received noise module 142 may flag the RF signals collected at the time stamp corresponding to background noise levels as potentially being inaccurate. In some embodiments, the received noise module 142 may compare RF signal data to background noise over time to improve the accuracy of the noise thresholds. The received radiation module may alert the user, such as with an audible beep or warning, a text message, or an alert to a connected mobile device. The alert would signal to the user that the current level of background noise is not conducive to getting an accurate measurement. The received noise module 142 may utilize the communication module 116 to communicate with the device network 104 in order to inform the network processing module 130 of the background noise data that corresponds with the received RF signal data. In this manner, the received noise module 142 may be simplified to just collect background noise data and allow the network processing module 130 to determine if the measure exceeded a threshold that would indicate the received RF signal data is too noisy to be relied upon for a blood glucose measurement, or if an alternative transfer function should be used to compensate for the noise.

Further, the system 100 may be configured to provide communication for transmitting and receiving signals between the monitoring device 102 and the device network 104. The device network 104 may comprise a network base module 126, a device network memory 128, and a network processing module 130.

The device network 104 may be configured to receive polling data from the device base module 120 using the communication module 116. The polling data of the device base module 120 may be transmitted to the network base module 126. Examples of the network base module 126 may include, but are not limited to, a means to control (not shown) the internet, a cloud network, a Wireless Fidelity (Wi-Fi) network, a Wireless Local Area Network (WLAN), Further, the device network 104 comprises the device network memory 128 configured to store the polling data received from the monitoring device 102. In one embodiment, the device network memory 128 may be configured to store the filtered RF signal received from the monitoring device 102. Examples of implementation of the device network memory 128 may include, but are not limited to, Cloud Storage, Cloud server, Random Access Memory (RAM), Read Only Memory (ROM), and/or a Secure Digital (SD) card. Further, the device network memory 128 may be configured to store an artificial intelligence (AI) correlation algorithm. Further, the network processing module 130 may be configured to perform different operations for processing the frequency signal to determine the user's blood glucose level.

The network processing module 130 may use real-time ground truth data to determine the user's blood glucose level. The real-time ground truth data may correspond to actual data related to health parameters in the blood sample of the user. For example, the real-time ground truth data related to the blood glucose level of Bob is 110 mg/dL, which could correspond to the received radio signal of frequency 122 GHz. In one embodiment, the real-time ground truth data may also correspond to a known value or standard parameter value to be obtained. For example, the known ground truth data related to the blood glucose level is 125 mg/dL for an average adult in the United States of America.

Further, an initial RF transmit signal TX is sent, which may be a measurement of the receive antenna RX signals, so these two signals are associated around the same time window. Generally, a series of RF Transmit TX signals are sent, and the associated RX receive antenna signals, which are stored right after another. The series of RF Transmit TX signals TX1, Tx2, TXn are different signals (frequencies and amplitudes). There are many possibilities of use cases regarding how many and at what variabilities RF Transmit TX signals are used.

Prior to the real-time use of the system, systematic tests are done to build a set of RF transmit signals that have associated receive RX antenna signals that can be analyzed, in which the analysis can be correlated to subjects that at the same time are taking ground truth blood samples for blood glucose. With a range of subjects with a range of blood glucose levels, this ground truth data is trained against the set of RF transmit signals that have associated receive RX antenna signals so that the data saved in the device network memory 128 is robust enough to be used as ground truth RX antenna signals (with their associated glucose levels) to correlate to newly obtained real-time RX antenna signals.

In order to start the process, the Transmit TX signals TX1, TX2, TXn are started from an initial TX1 signal, and the receive antenna signal associated with this TX1 signal is obtained. The initial TX1 signal is then updated to say TX2, and the process repeats until all transmit TX signals are sent.

Further, the initial or updated RF transmit signal may be sent to the one or more TX antennas 106. Further, the initial or updated RF signal may be received from the one or more RX antennas 108. The received initial or updated RF signal may be converted to a digital signal using the ADC 110. Further, the device network memory 128 may be configured to store the real-time ground truth data and the converted initial or updated RF signal. Also, device network memory 128 may be configured to store a correlation algorithm generated and executed by the network processing module 130. The network processing module 130 may be configured to execute the correlation algorithm between the real-time ground truth data and the converted initial or updated RF signal. In one embodiment, the network processing module 130 may be configured to execute the correlation algorithm between the real-time ground truth data and the filtered signal. Further, the network processing module 130 may be configured to determine whether the correlation is greater than or equal to a threshold limit. The threshold limit may correspond to a correlated value or coefficient between a derived value and a known value. In one embodiment, the threshold limit is less than 1. In another embodiment, the threshold limit is greater than or equal to 0.9. In one case, the network processing module 130 may determine that the correlation is greater than the threshold limit. In this case, the network processing module 130 may store the initial or updated the RF transmit signal in the device network memory 128. In another case, the network processing module 130 may determine that the Correlation is less than the threshold limit. In this case, the network processing module 130 may adjust the correlation algorithm. In one embodiment, the network processing module 130 may adjust the correlation algorithm using an auto-correlation technique, cross-correlation technique, convolutional correlation technique, positive correlation technique, negative correlation technique, and no correlation technique.

Successively, the network processing module 130 may determine whether the correlation reaches the threshold limit. In one case, the network processing module 130 may determine that the correlation reaches the threshold limit. In this case, the system 100 is configured to store the correlation algorithm in the device network memory 128. In another case, the network processing module 130 may determine that the Correlation algorithm does not reach the threshold limit. In this case, the network processing module 130 may update the initial or updated RF transmit signal and repeat until the correlation is greater than or equal to the threshold limit. Successively, the network processing module 130 is configured to store the best updated RF transmit signal and best correlation algorithm.

Further, the network base module 126 may be configured to poll transmit data to the cloud network 124 from the device base module 120. Further, the network base module 126 may store the received data to the device memory network 126. Successively, the network base module 126 may be configured to run the network processing module 130. The network base module 126 may determine whether a notification data exceeds the threshold limit. In one embodiment, the notification data may include information related to the best updated RF transmit signal. In this case, the network base module 126 may notify the monitoring device 102.

In one embodiment, the network processing module 130 is configured to read the received data from the device network memory 128. The network processing module 130 is configured to execute the best Correlation algorithm on the updated RF transmit signal. Further, the network processing module 130 may be configured to determine whether the threshold limit is acceptable. In this case, the network processing module 130 may send a notification signal to the monitoring device 102 via the network base module 126. In one embodiment, the notification signal sent to the monitoring device 102 may comprise the actual blood glucose level of the user. The user's actual blood glucose level may be displayed over the display unit 122 for the user.

FIG. 2 illustrates a posterior view 200 of a hand 202 of the user with an approximate location of a cephalic vein 204 and a basilic vein 206 overlaid/superimposed, according to an embodiment.

In one embodiment, wearable electronics like smartwatches and health and fitness trackers may often be worn on the wrist, like conventional wristwatches or rubber bands. It has been shown that the wrist's anatomy is significant for measuring glucose levels with active RF range radio waves. The cephalic vein 204 and basilic vein 206 may be seen superimposed on the back of a right hand or hand 202 in FIG. 2. The left-side measurement of a wrist's depth can yield a standard range of 40-60 mm (based on a wrist circumference in the range of 140-190 mm). The basilic vein 206 may be roughly located in subcutaneous tissue just under the skin.

It can be noted that the thickness of human skin in a wrist area is around 1-4 mm, and the thickness of the subcutaneous tissue may vary from 1-34 mm, although these thicknesses may vary based on many factors. It can be noted that the hand 202 includes both capillaries having a diameter in the range of 5-10 microns, and the cephalic vein 204 and the basilic vein 206 having a diameter range of 1-4 mm. The capillaries, the cephalic vein 204, and the basilic vein 206 may be approximately 1-9 mm below the skin of the hand 202. In one embodiment, the active RF radio frequency signals may be particularly employed in pinpointing the position of a blood artery like the basilic vein and thereby monitoring the blood glucose level.

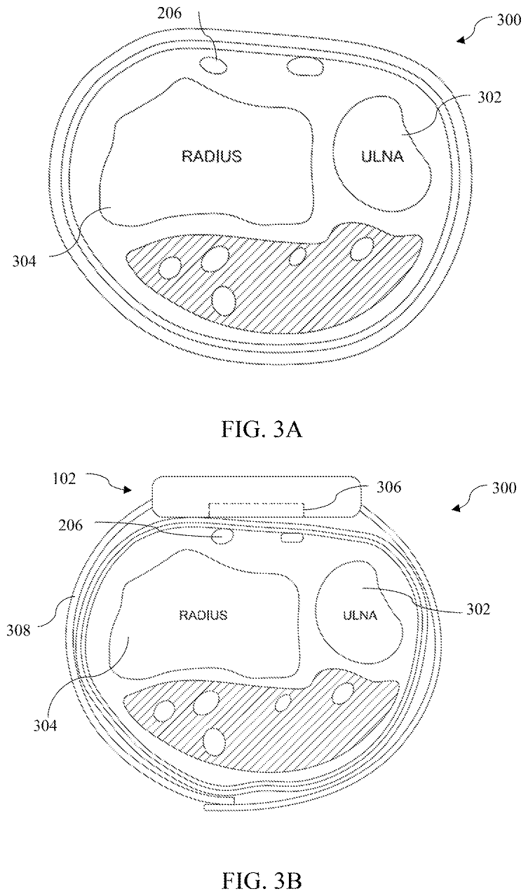

FIGS. 3A-3B illustrate a cross-sectional view of a wrist 300 with ulna bone 302 and the basilic vein 206, according to an embodiment.

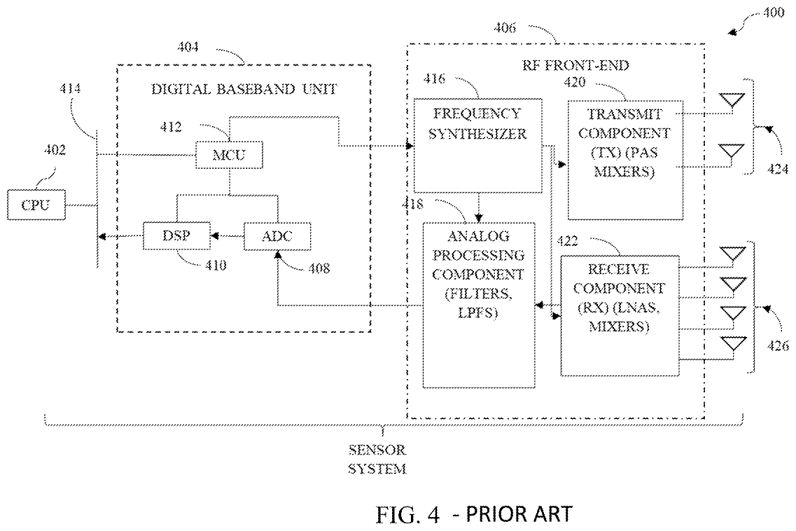

The wrist 300 may be provided with the monitoring device 102, as shown in FIG. 3B. In one example embodiment, the location of the monitoring device 102 relative to the wrist 300 and relative to the basilic vein 206 of the wrist 300 is depicted. The location of the monitoring device 102 relative to the anatomy of the wrist 300, including a radius bone 304, the ulna bone 302, and the basilic vein 206, is an important consideration in monitoring blood glucose levels using active RF radio frequency signals. Further, a dashed line block (shown by 306) represents an approximate location of a sensor system (not shown) on the monitoring device 102. The sensor system is described in conjunction with FIG. 4. Further, the monitoring device 102 may be provided with a strap 308 to tightly hold the monitoring device 102 around the wrist 300 in a secured position. It can be noted that the strap 308 may be provided with multiple fastening means (not shown) to adjust the monitoring device 102 around the wrist 300. The monitoring device 102 may be configured to transmit the active RF radio frequency signals towards the basilic vein 206 and receive the active RF radio frequency signals response from blood components inside the basilic vein 206. It can be noted that a large quantity of active RF radio frequency signals imparted underneath the skin of the wrist 300 may be response from the radius bone 304 and/or the ulna bone 302 in the wrist 300 as well as from some dense tissue, such as tendons and ligaments, that are located between the skin and the bones at a posterior of the wrist 300.

FIG. 4 illustrates a functional block diagram of a prior art sensor system 400 of the monitoring device 102 utilizing the active RF radio frequency signals to monitor the blood glucose level in the user, according to an embodiment.

The sensor system 400 may comprise a central processing unit (CPU) 402, a digital baseband unit 404, and a radio frequency (RF) front end 406. Further, the digital baseband unit 404 may comprise an analog-to-digital converter (ADC) 408, a digital signal processor (DSP) 410, and a microcontroller unit (MCU) 412. In one embodiment, the digital baseband unit 404 may include some other configurations, including some other combination of elements. The digital baseband unit 404 may be connected to the CPU 402 using bus connectors 414.

Further, the RF front-end 406 may comprise a frequency synthesizer 416, an analog processing component 418, a transmit (TX) component 420, and a receive (RX) component 422. Further, the TX component 420 may include PAS elements. The PAS elements correspond to power, amplifiers, and mixers. The RX component 422 may include LNAS elements. The LNAS elements correspond to low noise amplifiers (LNAs), variable gain amplifiers (VGAs), and mixers. The frequency synthesizer 416 may include elements to generate electrical signals at frequencies that are used by the TX component 420 and the RX components 422. In one embodiment, the frequency synthesizer 416 may include elements such as a crystal oscillator, a phase-locked loop (PLL), a frequency multiplier, and a combination thereof. The analog processing component 418 may include elements such as mixers and filters. In one embodiment, the filters may include low-pass filters (LPFs). In one embodiment, the frequency synthesizer 416, the analog processing component 418, the TX component 420, and the RX component 422 of the RF front end 406 may be implemented in hardware as electronic circuits that are fabricated on the same semiconductor substrate.

Further, the TX component 420 may comprise at least two TX antennas 424, and the RX component 422 may comprise at least four RX antennas 426. In one embodiment, the sensor system 400 may be provided with multiple TX antennas and RX antennas in a ratio of 1:2.

Further, the at least two TX antennas 424 and the at least four RX antennas 426 may be configured to transmit and receive active RF range radio frequency signals. In one embodiment, the sensor system 400, including the CPU 402, the digital baseband unit 404, and the RF front end 406 of the monitoring device 102, may be integrated into various configurations according to the size and shape of the monitoring device 102. For example, some configurations of components of the monitoring device 102 are fabricated on a semiconductor substrate and/or included in a packaged IC device or a combination of packaged IC devices. In one embodiment, the monitoring device 102 is designed to transmit and receive radio frequency signals at a pre-defined frequency. In one embodiment, the pre-defined frequency ranges between 122-126 GHz for 0.56-active RF radio frequency signals.

FIG. 5 illustrates a circuitry arrangement 500 of the one or more TX antennas 106 and the one or more RX antennas 108 of the sensor system 400 on a substrate 502 of the monitoring device 102, according to an embodiment.

The circuitry arrangement 500 may be fabricated on the substrate 502 with the one or more TX antennas 106 and the one or more RX antennas 108. Further, the circuitry arrangement 500 may be coupled to an input/output (I/O) interface 504. Further, the substrate 502 may have an outer footprint 506 and an inner footprint 508. The outer footprint 506 may correspond to an IC device of the monitoring device 102, and the inner footprint 508 may be a semiconductor substrate with circuits for the circuitry arrangement 500 and the one or more TX antennas 106 and the one or more RX antennas 108. The circuits are fabricated into the semiconductor substrate to conduct and process electrical signals transmitted by the one or more TX antennas 106 and received by the one or more RX antennas 108.

In one embodiment, the circuitry arrangement 500 with the inner footprint 508 and the outer footprint 506 has dimensions of a 2.5 mm radius. In one embodiment, the substrate 502 may have a footprint slightly smaller than the footprint of the circuitry arrangement 500. In one embodiment, the substrate 502 has approximately 0.1-1 mm dimensions less than the circuitry arrangement 500. In one exemplary embodiment, the circuitry arrangement 500 has a thickness of approximately 0.3-2 mm, and the substrate 502 has a thickness of about 0.1-0.7 mm. In one embodiment, the one or more TX antennas 106 and the one or more RX antennas 108 are designed specifically for radio frequency signals.

In one embodiment, the one or more TX antennas 106 and the one or more RX antennas 108 are depicted as square boxes of approximately 1 mm×1 mm and are linked on the same planar surface of the substrate 502 to the circuitry arrangement 500.

For example, the one or more TX antennas 106 and the one or more RX antennas 108 are attached on top surface of the circuitry arrangement 500 using a ceramic package material directly above the substrate 502 with conductive vias that electrically connect a conductive pad (not shown) of the substrate 502 to a transmission line of the one or more TX antennas 106 and the one or more RX antennas 108. In another embodiment, the one or more TX antennas 106 and the one or more RX antennas 108 may not have square arrangements. It can be noted that the square boxes correspond to an approximate footprint of the one or more TX antennas 106 and the one or more RX antennas 108. In one exemplary embodiment, the one or more TX antennas 106 and the one or more RX antennas 108 are microstrip patch antennas with dimensional functionality equivalent to the function of wavelength of the active RF radio frequency signals. In another exemplary embodiment, the one or more TX antennas 106 and the one or more RX antennas 108 correspond to antennas such as dipole antennas.

In one embodiment, the one or more TX antennas 106 and the one or more RX antennas 108 are positioned alternatively, with at least one TX antenna surrounded by at least two RX antennas. In another embodiment, the one or more TX antennas 106 and the one or more RX antennas 108 are positioned in an alternate series configuration connecting the circuitry arrangement 500. Further, each of the one or more TX antennas 106 on the left side of the circuitry arrangement 500 is positioned with at least two of the one or more RX antennas 108. Further, each one of the one or more TX antennas 106 on the right side of the circuitry arrangement 500 is positioned with at least two of the one or more RX antennas 108.

In one exemplary embodiment, each of the one or more TX antennas 106 includes channel-specific circuits (not shown) such as amplifiers, and each of the one or more RX antennas 108 includes channel-specific circuits (not shown) such as the mixers, the filters, and the LNAS, as described earlier. In another exemplary embodiment, the circuitry arrangement 500 includes a voltage control oscillator (VCO), a local oscillator (LO), frequency synthesizers, divider(s), mixers, ADCs, buffers, digital logic, DSPs, CPUs, and/or some combination thereof that may be utilized in conjunction with the channel-specific TX and RX antennas. Further, the one or more TX antennas 106 and the one or more RX antennas 108 may include an electrical interface (not shown) between a circuit on the substrate 502 and a corresponding antenna. In one embodiment, the electrical interface may be a conductive pad.

In one embodiment, the one or more TX antennas 106 and the one or more RX antennas 108 may be attached to the top surface of the substrate. It can be noted that the top surface may have a thickness of less than 0.5 mm. The one or more TX antennas 106 and the one or more RX antennas 108 may be connected to the electrical interface of each respective transmit/receive component separated by a fraction of a millimeter. Further, the substrate 502 may be integrated perpendicular to the plane of the circuitry arrangement 500 and connected to the electrical interface of each respective transmit/receive component. In one embodiment, multiple vias may be used when an antenna of the one or more TX antennas 106 or the one or more RX antennas 108 is provided with more than one transmission line. Such a collocated configuration of the substrate 502 enables a desired distribution of the one or more TX antennas 106 and the one or more RX antennas 108 to be maintained while effectively managing conductor losses in the sensor system 400. In one embodiment, the one or more RX antennas 108 may form a phased antenna array for health monitoring applications. It is desirable to have as much spatial separation as possible between the one or more RX antennas 108 to improve overall signal quality by obtaining unique signals from each RX antenna 108.

FIG. 6 illustrates the circuitry arrangement 500 of the one or more TX antennas 106 and the one or more RX antennas 108 of the sensor system 400 on the substrate 502 of the monitoring device 102 overlaid on the hand 202 of the user, according to an embodiment.

The circuitry arrangement 500, as explained in FIG. 5, is fabricated on the substrate 502 of the monitoring device 102 and overlaid on the wrist 300. The circuitry arrangement 500 may be oriented with respect to the basilic vein 206 and the cephalic vein 204, such that the one or more TX antennas 106 are configured with respect to the basilic vein 206 and the cephalic vein 204. In one embodiment, the one or more TX antennas 106 and RX antennas 108 may be distributed with respect to a direction of the basilic vein 206 and the cephalic vein 204.

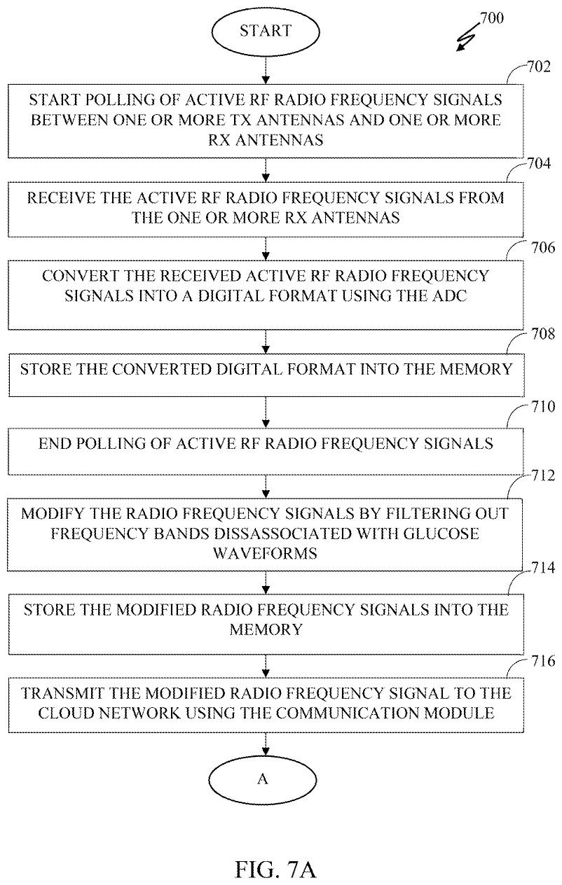

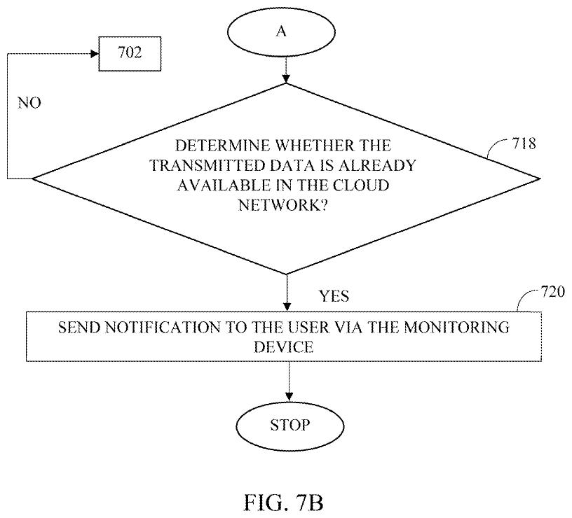

FIGS. 7A-7B illustrates a flowchart of a method 700 executed on the device base module 120, according to an embodiment.

At first, the device base module 120 may be configured to start polling the active RF radio frequency signals between the one or more TX antennas 106 and the one or more RX antennas at step 702. In one embodiment, the device base module 120 may be configured to read and process instructions stored in the memory 112 using the processor 114. For example, the device base module 120 sends 55-active RF radio signals of frequency range 120-126 GHz to a TX antenna and stores the 55-active RF radio signals into the memory 112. The TX antenna sends 55-active RF radio signals underneath a patient's skin.

Further, the device base module 120 may receive the active RF radio frequency signals from the one or more RX antennas 108 at step 704. For example, an RX antenna receives a response radio signal of frequency range 100-110 GHz from the patient's blood.

Successively, the device base module 120 may be configured to convert the received active RF radio frequency signals into a digital format using the ADC 110 at step 706. For example, the received radio signal of frequency range 100-110 GHz is converted into a series of 10-bit data signals. Successively, the device base module 120 may be configured to store converted digital format into the memory 112 at step 708.

Further, the device base module 120 may be configured to end polling of the active RF radio frequency signals at step 710. Successively, the device base module 120 may be configured to modify the radio frequency signals received from the one or more RX antennas 108 at step 712. In one embodiment, modifying the radio frequency signals may correspond to filtering out certain frequency bands that are not associated with glucose waveforms. The device base module 120 provides modified radio frequency signals containing frequency bands that correspond only to the glucose waveforms. For example, the device base module 120 modifies the radio signal of frequency range 100-110 GHz to a radio signal of frequency range 122-126 GHz.

Successively, the device base module 120 may be configured to store the modified radio frequency signals in the memory 112 at step 714. For example, the device base module 120 stores a radio signal of frequency range 122-126 GHz to the memory 112.

In another embodiment modifying a received antenna signal of a series of 10-bit data signals may be to amplify the signals to get the 10-bit data signals into a specified range. This is useful if there is high signal attenuation with too low signal amplitude.

In another embodiment modifying a received antenna signal of a series of 10-bit data signals may delete one of the 10-bit signals of the series if the 10-bit signal appears to be noise data. This is useful for improving signal-to-noise.

In another embodiment modifying a received antenna signal of a series of 10-bit data signals maybe to add interpolated signals to get the 10-bit data signals, for instance, to add a 10-bit data signal between two 10-bit data signals where the added signal is the average of the two data bit signals. This is useful if there is help normalizing a data series for the rate of change errors between 10-bit data signals.

Successively, the device base module 120 may be configured to transmit the modified active RF radio frequency signals to the cloud network 124 using the communication module 116 at step 716. For example, the device base module 120 transmits a radio signal of frequency range 122-126 GHz to the cloud network 124.

Further, the device base module 120 may be configured to determine whether the transmitted data is already available in the cloud network 124 at step 718. In one embodiment, the device base module 120, using the communication module 116, communicates with the cloud network to determine that the transmitted radio signal of frequency range 122-126 GHz is already available. In one case, the device base module 120 determines that the transmitted data is not already present in the cloud network 124. In this case, the device base module 120 may be redirected back to step 702 to poll the active RF radio frequency signals between the one or more TX antennas 106 and the one or more RX antennas 108. For example, the device base module 120 determines that the transmitted radio signal of frequency range 122-126 GHz is not present in the cloud network 124, and corresponding to the transmitted signal, there is no data related to the blood glucose level of the patient.

In another case, the device base module 120 determines that transmitted data is already present in the cloud network 124. For example, the device base module 120 reads cloud notification of the patient's blood glucose level as 110 mg/dL corresponding to a radio signal of 122-126 GHz frequency range. In this case, the device base module 120 may proceed to step 720 to notify the user via the monitoring device 102.

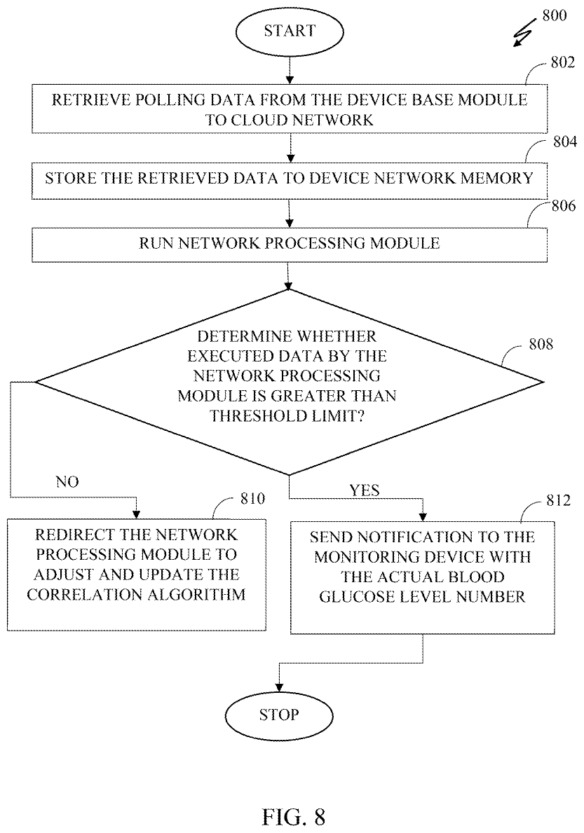

FIG. 8 illustrates a flowchart of a method 800 performed by the network base module 126, according to an embodiment.

At first, the network base module 126 may be configured to retrieve polling data from the device base module 120 to cloud network 124 at step 802. In one embodiment, the polling of the active RF radio frequency signals between the one or more TX antennas 106 and the one or more RX antennas 108, as mentioned in FIG. 7A, is retrieved by the network base module 126. For example, the network base module 126 retrieves a radio signal of frequency range 100-110 GHz converted into a 10-bit data signal from the device base module 120.

Successively, the network base module 126 may be configured to store the retrieved data to the device network memory 128 at step 804. For example, the network base module 126 stores a radio signal of frequency range 100-110 GHz converted into a 10-bit data signal to the device network memory 128.

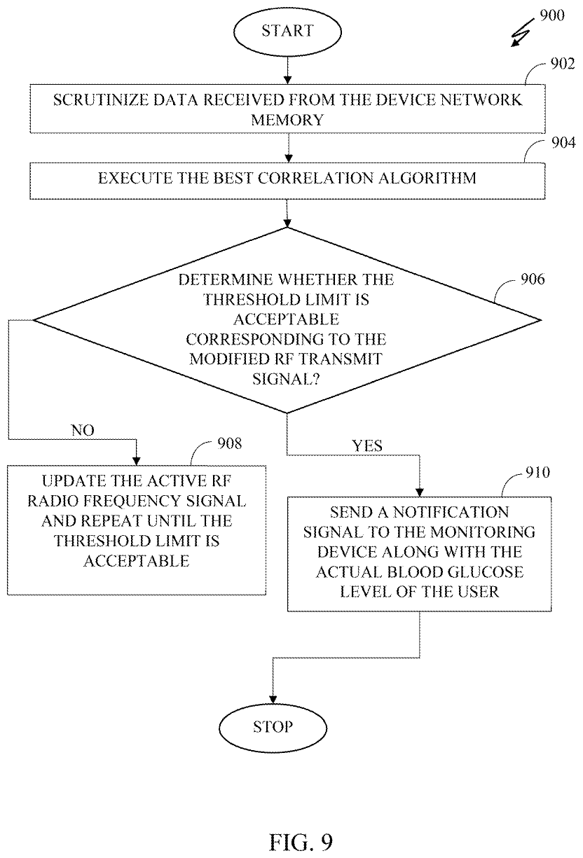

Further, the network base module 126 may be configured to run the network processing module 130, at step 806, for the stored best updated RF transmit signal and the best Correlation algorithm. The network processing module 130 is described later in conjunction with FIG. 9.

Successively, the network base module 126 may be configured to determine whether executed data by the network processing module 130 is greater than the threshold limit at step 808. In one embodiment, the network base module 126 may receive notification data once the network processing module 130 is operated. In another embodiment, the network base module 126 may be configured to determine the executed data when the network processing module 130 executes the best updated RF transmit signal and the best Correlation algorithm. In one case, the network base module 126 may determine that the executed data is less than the threshold limit. In this case, the network base module 126 may redirect the network processing module 130 to adjust and update the Correlation algorithm at step 810. For example, the network base module 126 determines that the executed data is 0.88, which is less than the threshold limit of 0.90.

In another case, the network base module 126 may determine that the executed data exceeds the threshold limit. In this case, the network base module 126 may proceed to step 812. For example, the network base module 126 determines that the executed data is 0.98, which is greater than the threshold limit of 0.90. Successively, the network base module 126 may notify the monitoring device 102 at step 812. For example, the network base module 126 sends a notification to the monitoring device 102 that the radio signal of frequency range 140-155 GHz is the best updated RF transmit signal corresponding to the threshold coefficient of 0.98 is greater than the threshold limit of 0.9.

FIG. 9 illustrates a flowchart of a method 900 performed by the network processing module 130, according to an embodiment.

At first, the network processing module 130 may be configured to scrutinize data received from the device network memory 128 at step 902. The network processing module 130 may be provided with the data to execute an operation for determining the health parameter of the user. In one embodiment, the network processing module 130 may determine the user's blood glucose level from the best updated RF transmit signal and the best Correlation algorithm stored in the memory 112 and the device network memory 128. For example, the network processing module 130 reads that the best updated RF transmit signal stored in the device network memory 128 is a radio signal of frequency range 140-155 GHz. The network processing module 130 may optionally, at step 902, receive data from, or execute the movement module to determine if movement of the monitoring device 102 exceeds a threshold, above which, the RF signal data is unreliable. The network processing module 130 may optionally, at step 902, receive data from, or execute the body temperature module to determine if the user's body temperature or the environmental temperature around the monitoring device 102 exceeds a threshold, above which, the RF signal data is unreliable. The network processing module 130 may optionally, at step 902, receive data from, or execute the body position module to determine if the position of the monitoring device 102 relative to the user exceeds a threshold, above which, the RF signal data is unreliable. The network processing module 130 may optionally, at step 902, receive data from, or execute the ECG module to determine if the user's cardiac data exceeds a threshold, above which, the RF signal data is unreliable. The network processing module 130 may optionally, at step 902, receive data from, or execute the circadian rhythm module to determine if the user's sleep data exceeds a threshold, above which, the RF signal data is unreliable. The network processing module 130 may optionally, at step 902, receive data from, or execute the received noise module to determine if the background noise around the monitoring device 102 exceeds a threshold, above which, the RF signal data is unreliable.

Successively, the network processing module 130 may be configured to execute the best Correlation algorithm at step 904. For example, the network processing module 130 runs a radio signal of frequency range 140-155 GHz.

Further, the network processing module 130 may be configured to determine whether the threshold limit is acceptable corresponding to the modified RF transmit signal at step 906. In one case, the network processing module 130 may determine that the threshold limit is unacceptable. In this case, the network processing module 130 may update the active RF radio frequency signals and repeat until the threshold limit is acceptable at step 908. For example, the network processing module 130 determines that the threshold limit is 0.87, which is less than an acceptable range of 0.90.

In another case, the network processing module 130 may determine that the threshold limit is acceptable. In this case, the network processing module 130 may proceed to step 910. For example, the network processing module 130 determines that the threshold limit is 0.92, which is slightly greater than the acceptable range of 0.90. Successively, the network processing module 130 may send a notification signal to the monitoring device 102 with the actual blood glucose level of the user at step 910. For example, the network processing module 130 sends that the radio signal of frequency range 140-155 GHz is acceptable to determine the user's blood glucose level is 110 mg/dL.

It will be appreciated by those skilled in the art that changes could be made to the exemplary embodiments described above without departing from the broad inventive concept thereof. It is to be understood, therefore, that this disclosure is not limited to the particular embodiments disclosed, but is intended to cover modifications within the spirit and scope of the subject disclosure as disclosed above.

1. A health monitoring system, comprising:
a monitoring device that includes one or more transmit antennas configured to transmit radio-frequency (RF) analyte detection signals into a user and one or more receive antennas configured to detect RF analyte signals that result from the RF analyte detection signals transmitted into the user;
an analog-to-digital converter connected to the one or more receive antennas and receiving the RF analyte signals detected by the one or more receive antennas; and
a motion sensor that detects motion of the user during transmission of the RF analyte detection signals by the one or more transmit antennas and during detection of the RF analyte signals by the one or more receive antennas.

2. The health monitoring system of claim 1, further comprising a body temperature sensor that detects the temperature of the user during transmission of the RF analyte detection signals by the one or more transmit antennas and during detection of the RF analyte signals by the one or more receive antennas.

3. The health monitoring system of claim 2, wherein the analog-to-digital converter, the motion sensor and the body temperature sensor are part of the monitoring device.

4. The health monitoring system of claim 1, further comprising at least one of the following:
a) a body position sensor that senses the user's body position during transmission of the RF analyte detection signals by the one or more transmit antennas and during detection of the RF analyte signals by the one or more receive antennas;
b) an electrocardiogram sensor that senses the user's heartbeat during transmission of the RF analyte detection signals by the one or more transmit antennas and during detection of the RF analyte signals by the one or more receive antennas;
c) a circadian rhythm sensor that senses circadian data of the user during transmission of the RF analyte detection signals by the one or more transmit antennas and during detection of the RF analyte signals by the one or more receive antennas;
d) a background noise sensor that senses background noise around the user during transmission of the RF analyte detection signals by the one or more transmit antennas and during detection of the RF analyte signals by the one or more receive antennas.

5. The health monitoring system of claim 4, comprising two or more of a)-d); three or more of a)-d); or each of a)-d).

6. The health monitoring system of claim 2, further comprising a memory that stores a motion threshold and a body temperature threshold.

7. A health monitoring system, comprising:
a monitoring device that includes one or more transmit antennas configured to transmit radio-frequency (RF) analyte detection signals into a user and one or more receive antennas configured to detect RF analyte signals that result from the RF analyte detection signals transmitted into the user;
an analog-to-digital converter connected to the one or more receive antennas and receiving the RF analyte signals detected by the one or more receive antennas; and
a body temperature sensor that detects the temperature of the user during transmission of the RF analyte detection signals by the one or more transmit antennas and during detection of the RF analyte signals by the one or more receive antennas.

8. The health monitoring system of claim 7, wherein the analog-to-digital converter and the body temperature sensor are part of the monitoring device.

9. The health monitoring system of claim 7, further comprising at least one of the following:
a) a body position sensor that senses the user's body position during transmission of the RF analyte detection signals by the one or more transmit antennas and during detection of the RF analyte signals by the one or more receive antennas;
b) an electrocardiogram sensor that senses the user's heartbeat during transmission of the RF analyte detection signals by the one or more transmit antennas and during detection of the RF analyte signals by the one or more receive antennas;
c) a circadian rhythm sensor that senses circadian data of the user during transmission of the RF analyte detection signals by the one or more transmit antennas and during detection of the RF analyte signals by the one or more receive antennas;
d) a background noise sensor that senses background noise around the user during transmission of the RF analyte detection signals by the one or more transmit antennas and during detection of the RF analyte signals by the one or more receive antennas.

10. The health monitoring system of claim 9, comprising two or more of a)-d); three or more of a)-d); or each of a)-d).

11. The health monitoring system of claim 7, further comprising a memory that stores a body temperature threshold.

12. A health monitoring method, comprising:
detecting an analyte in a user by transmitting radio-frequency (RF) analyte detection signals into the user from one or more transmit antennas and detecting, using one or more receive antennas, RF analyte signals that result from the RF analyte detection signals transmitted into the user;
converting the detected RF analyte signals from analog signals to digital signals using an analog-to-digital converter connected to the one or more receive antennas; and at least one of the following:
a) during transmission of the RF analyte detection signals by the one or more transmit antennas and during detection of the RF analyte signals by the one or more receive antennas, sensing motion of the user using a motion sensor;
b) during transmission of the RF analyte detection signals by the one or more transmit antennas and during detection of the RF analyte signals by the one or more receive antennas, sensing a body temperature of the user using a body temperature sensor.

13. The health monitoring method of claim 12, comprising a), and comparing the sensed motion of the user to a motion threshold stored in a memory.

14. The health monitoring method of claim 12, comprising b), and comparing the sensed body temperature of the user to a body temperature threshold stored in a memory.

15. The health monitoring method of claim 12, comprising a) and b); and comparing the sensed motion of the user to a motion threshold stored in a memory, and comparing the sensed body temperature of the user to a body temperature threshold stored in the memory.

16. The health monitoring method of claim 12, further comprising at least one of the following:
a) during transmission of the RF analyte detection signals by the one or more transmit antennas and during detection of the RF analyte signals by the one or more receive antennas, sensing the user's body position using a body position sensor;
b) during transmission of the RF analyte detection signals by the one or more transmit antennas and during detection of the RF analyte signals by the one or more receive antennas, sensing the user's heartbeat using an electrocardiogram sensor;
c) during transmission of the RF analyte detection signals by the one or more transmit antennas and during detection of the RF analyte signals by the one or more receive antennas, sensing circadian data of the user using a circadian rhythm sensor; and
d) during transmission of the RF analyte detection signals by the one or more transmit antennas and during detection of the RF analyte signals by the one or more receive antennas, sensing background noise around the user using a background noise sensor.

17. The health monitoring method of claim 16, comprising two or more of a)-d); three or more of a)-d); or each of a)-d).

18. The health monitoring method of claim 16, comprising each of a)-d); and further comprising comparing the sensed body position of the user to a body position threshold; comparing the sensed user heartbeat to a heartbeat threshold; comparing the sensed circadian data to a circadian rhythm threshold; and comparing the sensed background noise to a background noise threshold.

19. The health monitoring method of claim 12, further comprising modifying the digital signals by filtering out frequency bands not associated with glucose waveforms.
